(19) United States
(12) Patent Application Publication
(10) Pub. No.: US 2010/0262229 A1
(43) Pub. Date: Oct. 14, 2010

(54) Endoprosthesis and Method for Manufacturing Same

(75) Inventor: Roland Rohde, Burgdorf (DE)

Correspondence Address: BIOTECH BEACH LAW GROUP , PC, 5677 OBERLIN DRIVE, SUITE 204, SAN DIEGO, CA, 92121, US

(73) Assignee: BIOTRONIK VI PATENT AG, Baar (CH)

(21) Appl. No.: 12/509,646

(22) Filed: Jul. 27, 2009

(30) Foreign Application Priority Data
Jul. 28, 2008 (DE) 10 2008 040 791.7

(51) Int. Cl. A61F 2/82

(52) U.S. Cl. 623/1.46

(57) ABSTRACT

The invention relates to intraluminal endoprosthesis, preferably a stent consisting essentially of a base body and optionally a coating covering the surface of the base body at least partially. The invention is characterized in that the base body and/or the coating has at least one compound from the group of polyphosphates, magnesium oxyhalides, preferably Sorel cement. Furthermore, a method is described for manufacturing such an intraluminal endoprosthesis.

### CROSS-REFERENCE TO RELATED APPLICATIONS

This application claims benefit of priority to German patent application number DE 10 2008 040 791.7, filed on Jul. 28, 2008; the contents of which are herein incorporated by reference in their entirety.

### FIELD OF THE INVENTION

The invention relates to an intraluminal endoprosthesis, preferably a stent.

### BACKGROUND OF THE INVENTION

Endoprostheses are prostheses and implants that remain permanently in the body. Stents (vascular supports) are especially important representatives of the intraluminal endoprostheses. Stents are endovascular prostheses, which may be used for treatment of stenoses (vascular occlusions). They have a tubular or hollow cylindrical base body, preferably consisting of webs folded in a zigzag or meandering pattern, running essentially in the circumferential direction, as supporting elements and also webs running longitudinally connecting these supporting elements as connecting struts. The base body may be provided at least partially with a coating and is open at both ends in the axial direction of the hollow cylinder. Such an endoprosthesis is inserted into the vessel to be treated by means of a catheter, for example, and serves to support the vessel. Through the use of stents, stenosed areas in the vessels can be dilated, resulting in a larger lumen.

After insertion of a stent into a vessel, there is a risk of a restenosis, i.e., reocclusion of the stented vascular area, because of various processes, e.g., coagulation of body fluid or occlusion in this area caused by neointima formation due to a change in flow or an inflammation process.

One possibility for solving this problem is to manufacture the stent from a biodegradable material. Biodegradation is understood to refer to hydrolytic, enzymatic and other metabolic degradation processes in a living organism, caused mainly by body fluids coming in contact with the endoprosthesis and leading to gradual dissolution of at least large portions of the endoprosthesis. The term “biocorrosion” is often used as a synonym for the term “biodegradation.” The term “bioresorption” comprises subsequent resorption of degradation products by the living organism.

Materials suitable for the base body of biodegradable endoprostheses may be of a polymeric or metallic type, for example.

EP 1 270 023 B1 discloses such a stent made of an in-vivo degradable (by corrosion) metallic material and is formed from an alloy containing magnesium as the main ingredient. This material comprises in particular 79-97% magnesium, 2-5% aluminum, 0-12% lithium and 1-4% rare earths, in particular cerium, lanthanum, neodymium and/or praseodymium. Magnesium is tolerated very well physiologically and the expected degradation rate can be controlled through a suitable choice of the other alloy components. Based on inhomogeneities in the material, however, locally accelerated corrosion may occur with base metals in particular such as magnesium, resulting in premature loss of mechanical integrity and stability of the implant. Furthermore, bacterial colonization may occur on the surfaces of implants, possibly serving as a source of infection and even leading to disturbances in coagulation processes in the case of stents, in particular leading to fibrin deposits and thromboses. Another disadvantage of implants made of lightweight metals in particular is the lack of radiopacity, which prevents effective tracking and follow-up monitoring of the implantation process.

DE 103 08 186 B4 discloses an antimicrobial phosphate glass having numerous components, in particular 66-80 wt % P₂O₅. The glass composition has a high chemical stability and a high reactivity and may be used in the fields of medicine and cosmetics. However, the composition defined in the publication cited above is not suitable as the base body material or coating material for intraluminal endoprostheses because it has toxic or potentially toxic components with Al₂O₃ and the Cu, Ge, Te, Cr and F compounds listed.

Another problem with a stented vessel is that degradable, i.e., biodegradable, stents may undergo a loss of integrity too soon due to initial degradation proceeding too quickly. Furthermore, such medical implants tend to develop bacterial contamination due to their method of use or to a predisposition of a specific patient group, endangering the patient and necessitating repeat surgery/antibiotic therapy. Furthermore, it is desirable if stents or other intraluminal endoprostheses are radiopaque, allowing the implant to be localized during introduction or in the course of further treatment.

### SUMMARY OF THE INVENTION

The feature of the present invention is to create an intraluminal endoprosthesis which prevents restenosis of the stented vessel due to neointima formation and ensures integrity over a longer period of time. Furthermore, this object consists of providing a simple and inexpensive method for manufacturing an intraluminal endoprosthesis.

The feature defined above is achieved by an intraluminal endoprosthesis whose base body and/or coating which at least partially covers the surface of the base body has at least one compound from the group of polyphosphates, magnesium oxyhalides or preferably Sorel cement.

The aforementioned compounds meet the object defined above.

The advantage of an inventive intraluminal endoprosthesis with polyphosphates consists of the fact that polyphosphates as a component of the base body and/or a coating prevent restenosis through prompt biodegradation of the base body. A coating with polyphosphates, as a coating of a degradable stent, e.g., an AMS (absorbable metal stent), prevents the progress of degradation by delaying water access, and in the case of a magnesium stent, by actively influencing the surface pH. Furthermore, polyphosphates are microbicidal.

A restenosis can also be prevented by prompt biodegradation of the stent body in the case of intraluminal endoprostheses having a base body or a coating containing magnesium oxyhalides (also known as magnesium oxide halides), preferably Sorel cement (also known as magnesia cement, Sorel's cement or magnesite binder, composition: MgCl₂×3 Mg(OH)₂×8 H₂O).

It is advantageous in particular that the mechanical properties of Sorel cement and other magnesium oxyhalides and/or polyphosphates can be controlled by additives. Additives suitable for controlling mechanical properties include in particular proteins such as gelatin, albumin and fibrin; polymers such as polyphosphates, polylactates, polyhydroxybutyric acid, hyaluronic acid, cellulose and other polysaccharides and contrast agents containing iodide; phosphates, iodine. The amount of additives by weight may constitute up to 50% of the resulting composite or up to 80% in the case of granular additives.

Sorel cement advantageously contains only components that occur in blood physiologically, so Sorel cement is biocompatible. Sorel cement adheres to metallic surfaces and many other surfaces, preventing water diffusion, and is degraded in aqueous solutions, so it is especially suitable as a coating. The degradation of Sorel cement can also be retarded by a subsequent treatment with phosphoric acid or soluble phosphates (phosphating). On addition of hydrophobic admixtures, e.g., hydrophobic polymers, PTFE, fatty acid esters, cholesterol esters, waxes, phosphate esters and/or ethyl silicate esters, the hydrophilicity and thus also the water solubility can be reduced.

A preferred composition of a base body and/or a coating with polyphosphates and optionally also magnesium oxyhalides preferably contains the following ingredients (all the following percentage amounts are percent by weight, unless otherwise indicated): P₂O₅ approximately 30-100%, ZnO approximately <1%, alkali compounds approximately >1%, Na₂O approximately 0-5%. K₂O approximately 0-15%, Li₂O approximately 0-15%, AgI approximately 0-15%, MgI₂ approximately 0-20%, MgCl₂ approximately 0-30%, MgO approximately 0-60%. Al₂O₃ approximately <1%. The composition given above is based on the anhydrous mineral content.

Furthermore, it is advantageous to vary the stoichiometry of the components MgO (and/or Mg(OH)₂) and MgCl₂ in the Sorel cement of the base body and/or the coating, so that a deficit or excess of MgO develops. This makes it possible to additionally control interfacial pH levels and degradation rates of composite materials beyond the influence of the additives. In this context, an MgCl₂/Mg(OH)₂ ratio of approximately 1:1 to 1:7, in particular from approximately 1:3 to 1:6, is especially advantageous.

For use as a radiopaque material in the base body or the coating, MgCl₂ in the Sorel cement can be replaced entirely or partially by MgI₂, so the empirical formula is:

[(1−a) MgCl₂ a MgI₂]×b Mg(OH)₂×c H₂O, where 0<a≦1, 1<b≦7, c>1. (Formula 1)

In a preferred exemplary embodiment, the base body and/or the coating is/are provided with at least one active pharmaceutical substance and/or a radiopaque substance, preferably at least one element or compound from the group of
biopolymers, preferably proteins, peptides, amino acids, nucleic acids, polysaccharides,
lipids, preferably fatty acids, fatty acid esters, waxes, hydrophobic compounds, preferably cholesterol esters, phosphate esters, ethyl silicate esters,
radiopaque elements or compounds, preferably noble metals.

An “active pharmaceutical substance” (or a pharmaceutically active substance or a therapeutically or pharmacologically active substance) in the sense of the present invention is a plant-based, animal or synthetic active ingredient (medication), which is used in a suitable dose as a therapeutic agent to influence states or functions of the body, as a substitute for active ingredients produced naturally by the human or animal body and to eliminate disease pathogens or exogenous substances or render them harmless. Release of the substance in the endoprosthesis environment has a positive effect on the course of healing and/or counteracts physiological tissue changes due to a surgical procedure.

Such active pharmaceutical substances have an anti-inflammatory and/or antiproliferative and/or spasmolytic effect, so that restenoses, inflammations or (vascular) spasms, for example, can be prevented. In other exemplary embodiments, such substances may consist of one or more substances from the active ingredient group of the compounds or elements listed above or calcium channel blockers, lipid regulators (e.g., fibrates), immunosuppressants, calcineurin inhibitors (e.g., tacrolimus), antiphlogistics (e.g., cortisone or diclofenac), anti-inflammatories (e.g., imidazoles), antiallergics, oligonucleotides (e.g., dODN), estrogens (e.g., genistein), endothelializing agents (e.g., fibrin), steroids, proteins, peptides, vasodilators (e.g., sartans), antiproliferative substances of the taxols or taxanes, preferably paclitaxel or sirolimus here and derivatives thereof as well as from such lipophilic substances, which inhibit tissue calcification or neointima formation, such as vitamin A and D derivatives and phylloquinone/menaquinone (vitamin K) derivatives.

Active pharmaceutical substances can be introduced into a Sorel cement matrix by adding one or more solutions or slurries of the additives to be added to one or both Sorel cement components (dry or slurried MgO/Mg(OH)₂ and dry or dissolved MgCl.6H₂O) in production of the Sorel cement matrix. Before production of the cement in particular, granular additives, for example, polymer beads or protein globules containing the active pharmaceutical substance and/or the radiopaque substance may be mixed with one of the Sorel components or secured on the stent base body before applying the Sorel matrix.

In another preferred exemplary embodiment, the endoprosthesis has a base body comprising a metal alloy and/or a polymer and/or a polyphosphate and/or a Sorel cement plus a coating comprising the Sorel cement, whereby the coating has a layer thickness of at least approximately 5 μm, preferably at least approximately 50 μm, max, approximately 200 p.m. With a suitable MgCl:Mg(OH)₂ stoichiometry, the Sorel cement coating produces an alkaline medium that reduces the corrosive attack by body fluids on the supporting base body.

The chemical/physical properties and degradation can be controlled especially preferably by providing the endoprosthesis at least partially with a polyphosphate coating having a layer thickness of at least 5 μm, preferably at least approximately 30 μm, max, approximately 200 μm, whereby the molecules of the polyphosphate coating preferably have a chain length of at least ten phosphate groups.

In another exemplary embodiment, a biodegradable metal stent, preferably of a magnesium alloy, is provided with a polyphosphate layer with a P₂O₅ content of 85 wt % and 5 wt % Na₂O, 5 wt % AgI, 5 wt % MgO, based on the anhydrous mineral content, in a layer thickness of at least approximately 30 μm and then rinsed with an MgI₂ solution. In rinsing, there is an exchange of Na ions.

Production of an intraluminal endoprosthesis in which the coating containing at least one compound from the group of polyphosphates and magnesium oxyhalides is applied by a dipping or spray method is especially inexpensive. For example, a pulverized form of long-chain water-insoluble sodium polyphosphate may be mixed dry with MgO and then applied to the endoprosthesis in a slurried form by dipping or spraying. A Sorel cement layer can be produced by spraying simultaneously with or after drying by dipping in a concentrated aqueous solution containing magnesium iodide and chloride. This operation is may be repeated until reaching a layer thickness of approximately 20 μm to approximately 30 μm. Next by phosphating and by ion exchange, a reduction in the solubility and pH in the direction of the neutral range can be achieved at the surface.

It is also preferable if the coating is produced by treatment with a mixed alkali/alkaline earth halide or hydroxide solution. Sorel cement layer can be produced in situ in this way.

The solubility of the surface of the coating can be reduced advantageously by additionally treating the surface with a phosphoric acid and/or phosphate solution and/or with a water-repellent sealant, preferably a wax and/or a fat. These phosphoric acid and/or phosphate solutions have a pH in the neutral or slightly acidic range (phosphate buffer). Mixtures of alkali hydrogen phosphate and alkali dihydrogen phosphates are preferably used here.

An especially advantageous method of producing an intraluminal endoprosthesis consists of coating the base body of the endoprosthesis with insoluble polyphosphate powder and then melting it superficially, e.g., by laser processing. The coating is easily applied by the inventive method. A dense polyphosphate melt is formed by superficial melting of the powder.

Additional goals, features, advantages and possible applications of the invention are derived from the following description of an inventive intraluminal endoprosthesis as well as a production process for such an endoprosthesis on the basis of figures. All the features described and/or illustrated graphically here, either alone or in any combination, may form the subject matter of the present invention, even independently of how they are combined in the individual claims or how they refer back to previous claims.

### BRIEF DESCRIPTION OF THE DRAWINGS

Schematically in the drawings:

FIG. 1 shows a cross section through an extruded tube representing a semifinished product for production of an inventive endoprosthesis, and

FIGS. 2 and 3 show a cross section through a first injection molding device and

FIG. 4 shows a cross section through a second injection molding device, which together serve to produce an inventive endoprosthesis.

### DETAILED DESCRIPTION OF THE INVENTION

### Example 1

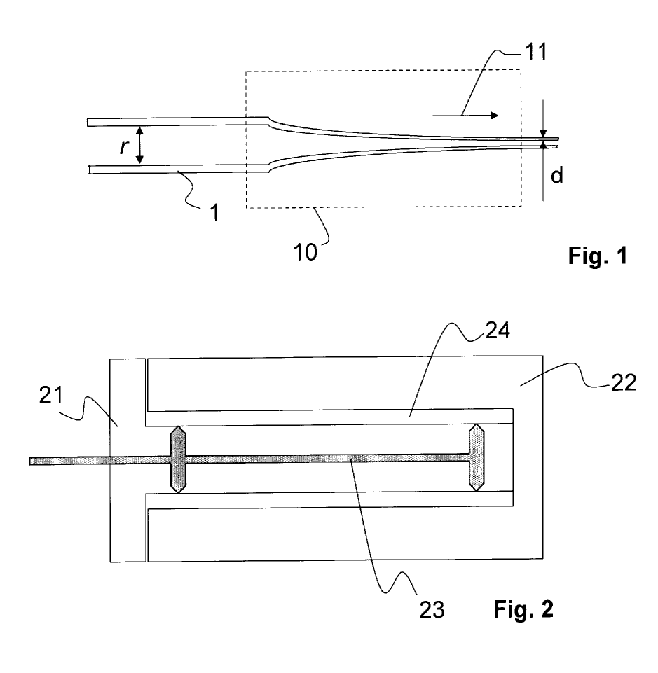

For production of a stent, a melt is first prepared from a mixture of pulverized polyphosphate and additives having a composition comprising approximately 87 wt % water-insoluble phosphate glass with a P₂O₅ content of approximately 65-70 wt % and 13 wt % silver iodide, and the thin-walled tube 1 shown in cross section in FIG. 1 is extruded with an inside diameter r of approximately 5 mm and a wall thickness of 0.7 mm, for example. This tube can be drawn out further manually or in a device 10 to form a tube with even thinner walls of a suitable wall thickness d, e.g., a wall thickness d of approximately 150 μm, if necessary, with a suitable temperature program, e.g. by heating over a Bunsen burner until the material becomes drawable. The direction of pulling 11 in the pulling device 10 runs in the direction of the longitudinal axis of the tube. According to the properties typical of the tube material, a suitable stent cutting pattern (design) is drafted with the help of a computer program based on the method of finite elements. Using a laser, a stent base body is cut out of the thin-walled phosphate tube and then smoothed by polishing. An ion exchange procedure may then be performed, if necessary.

### Example 2

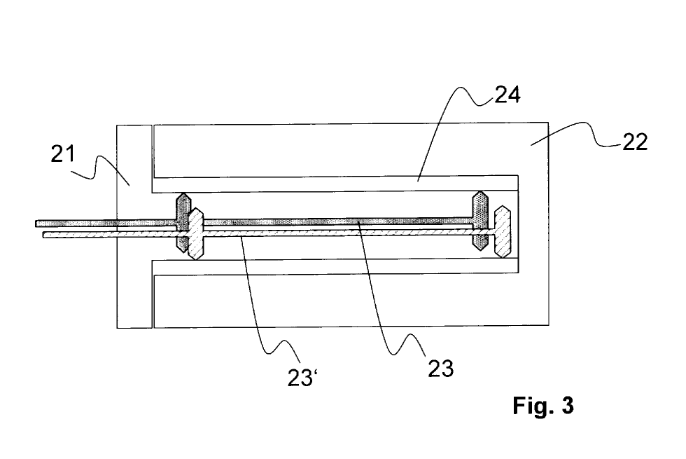

In a suitable injection molding device with the molded parts 21 and 22 stuck one inside the other as illustrated in FIGS. 2 and 3, a thin-walled tube (hollow cylinder) is shaped from an inventive Sorel cement mixture having a low water content. The hollow cylinder is formed by the hollow cylindrical cavity area 24, which is created between the hollow cylindrical molded part 22 (with a bottom) and the double-cylindrical molded part 21. The Sorel cement mixture is arranged together with additives containing polyphosphates and/or other radiopaque and/or active pharmaceutical substances and/or substances which improve the mechanical properties, e.g., pharmacologically active substances of the substance classes listed above embedded in biopolymers, are arranged in the cavity area 24. By injection of water (H₂O) and/or a solution of additives (e.g. MgCl₂ solution) into one or more injection channel systems (reference numerals 23 and 23′ in FIGS. 2 to 3), additives are added to the tube, which consists essentially of dry solids, or the hardening process is initiated. The fully hardened tube is then subjected to a stent cutting procedure similar to that with the tube from Example 1. Then again, a polishing, a phosphating and/or another loading with pharmacologically active substances or substances that control the degradation may then be performed, e.g., impregnation with water-insoluble or pharmacologically active lipids. Furthermore, after shaping or after the cutting procedure, a thermal treatment, e.g., heating to approximately 45° C. up to approximately 450° C. may then be performed to sinter or homogenize the material.

### Example 3

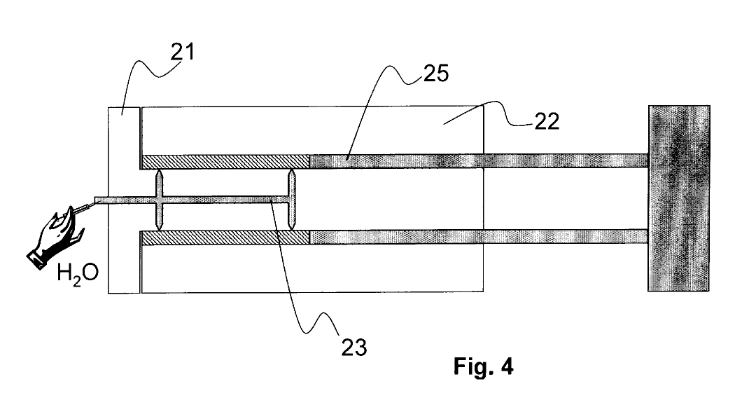

In a device according to FIG. 4, a homogenized mixture of powdered anhydrous constituents having the inventive stoichiometry consisting, for example, of approximately 55 wt % of a mixture of MgO, MgCl₂ (anhydrous), MgI, (anhydrous) mixture according to formula 1 and a water-insoluble polyphosphate with a P₂O₅ content of approximately 70 wt % is compressed at a pressure of approximately 100 kg/cm² (or more) with the help of the tube ram 25 in the tubular cavity 24 formed by the molds 21 and 22 and/or is sintered at temperatures up to approximately 450° C. and then mixed with purified water or a salt solution until the Sorel cement is formed or solidified by subsequent heating and/or moistening to form a stable stent base body. The injection molding device in FIG. 4 corresponds in design to the device shown in FIGS. 2 and 3 except for the tube ram 25.

It will be apparent to those skilled in the art that numerous modifications and variations of is the described examples and embodiments are possible in light of the above teaching. The disclosed examples and embodiments are presented for purposes of illustration only. Therefore, it is the intent to cover all such modifications and alternate embodiments as may come within the true scope of this invention.

### LIST OF REFERENCE NUMERALS

1 tube
10 drawing device
11 drawing direction
21, 22 injection mold, e.g., of metal
23, 23′ injection channel with injection nozzles at the end
24 cavity area
25 tubular pressure ram for compressing the material in the mold area 24
d wall thickness of the drawn tube 1
r inside diameter of the extruded tube 1

What is claimed is:

1. An intraluminal endoprosthesis, preferably a stent, consisting essentially of a base body and optionally a coating that covers the surface of the base body at least partially, characterized in that the base body and/or the coating comprises at least one compound from the group of
polyphosphates, magnesium oxyhalides, preferably Sorel cement.

2. The intraluminal endoprosthesis according to claim 1, characterized in that the base body and/or the coating is provided with at least one active pharmaceutical substance and/or radiopaque substance, preferably at least one element or one compound from the group of
biopolymers, preferably proteins, peptides, amino acids, nucleic acids, polysaccharides.
lipids, preferably fatty acids, fatty acid esters, waxes,
hydrophobic compounds, preferably cholesterol esters, phosphate esters, ethyl silicate esters,
radiopaque elements or compounds, preferably noble metals, metal salts, iodine compounds.

3. The intraluminal endoprosthesis according to claim 1, characterized in that the base body and/or the coating comprises Sorel cement with a deficit or excess of MgO.

4. The intraluminal endoprosthesis according to claim 1, characterized in that the base body and/or the coating has a magnesium oxy halide with
[(1−a) MgCl₂ a MgI₂]×b Mg(OH)₂×c H₂O, where 0<a≦1, 1<b≦7, c>1.

5. The intraluminal endoprosthesis according to claim 1, characterized in that the endoprosthesis comprises a base body having a metal alloy and a coating containing Sorel cement, whereby the coating has a layer thickness of at least approximately 5 μm, preferably at least approximately 50 μm and max. approximately 200 μm.

6. The intraluminal endoprosthesis according to claim 1, characterized in that the endoprosthesis is provided at least partially with a polyphosphate coating, which comprises a layer thickness of at least approximately 5 μm, preferably at least approximately 30 μm and max. approximately 200 μm, whereby the molecules of the polyphosphate coating preferably have a chain length of at least 10 phosphate groups.

7. The intraluminal endoprosthesis according to claim 1, characterized in that the base body and/or the coating has at least approximately 30 wt % P₂O₅ which forms a polyphosphate, based on the anhydrous mineral content of the base body and/or the coating.

8. The intraluminal endoprosthesis according to claim 1, characterized in that the coating is applied by means of a dip or spray method.

9. The intraluminal endoprosthesis according to claim 1, characterized in that the magnesium oxyhalide coating is produced by means of a treatment with a mixed alkali/alkaline earth halide or hydroxide solution.

10. The intraluminal endoprosthesis according to claim 1, characterized in that the surface of the magnesium oxyhalide coating is additionally provided with a phosphoric acid solution and/or a phosphate solution and/or with a water-repellent sealant, preferably with a wax and/or another lipophilic substance.

11. The method for manufacturing an intraluminal endoprosthesis according to claim 1, characterized in that the base body is coated at least partially with insoluble polyphosphate powder, preferably by means of a dip or spray method and then melted superficially.
